(19) United States
(12) Patent Application Publication
(10) Pub. No.: US 2008/0182235 A1
(43) Pub. Date: Jul. 31, 2008

(54) Detection of Analytes in Samples Using Liposome-Amplified Luminescence and Magnetic Separation

(75) Inventors: Andrew Hearn, Chicago, IL (US); Judith Madden, Valencia, CA (US); Subramani Sellappan, Aurora, IL (US); Antje J. Baeumner, Ithaca, NY (US); Natalya V. Zaytseva, Painted Post, NY (US)

Correspondence Address: LOEB & LOEB, LLP, 321 NORTH CLARK, SUITE 2300, CHICAGO, IL, 60610-4746, US

(73) Assignees: Celsis International plc, Chicago, IL (US); Cornell University, Ithaca, NY (US)

(21) Appl. No.: 11/669,078

(22) Filed: Jan. 30, 2007

(51) Int. Cl. C12Q 1/70; C12Q 1/48; G01N 33/53; G01N 33/554; C12Q 1/68

(52) U.S. Cl. 435/5; 435/15; 435/6; 435/7.31; 435/7.32

(57) ABSTRACT

The invention relates to the encapsulation of luminescence-related molecules, including but not limited to, adenosine triphosphate (ATP), adenylate kinase (AK), alkaline phosphatase (ALP), luminol and luciferin/luciferase cocktails, within liposomes. These liposomes can be employed to enhance the luminescence detection of microorganisms and compounds in various products and samples. The liposomes containing the luminescence-related molecules can bear a probe which has a specific sequence or structure that, in turn can be used to hybridize to, or couple with, a portion of the target analyte. Within the same assay, paramagnetic beads can bear a probe having a specific sequence or structure that, can hybridize to, or couple with, a second portion of the target analyte to create a complex of analyte bound to paramagnetic beads and liposomes. This type of assay can be often referred to as a ‘sandwich’ assay. Once the probes hybridize to, or couple with, their targets, a complex can be formed of the paramagnetic beads, the analyte, or portion thereof, and the liposomes. This complex can then be washed to remove those components that are non-hybridized or non-coupled. Then, the paramagnetic bead-analyte-liposome complexes can be isolated from the sample using magnetic separation techniques and can be treated so as to release their encapsulated ATP, AK or other luminescence-related compounds. The resulting luminescence can then be determined in a chemical assay. This determination can be qualitative (i.e., an absence/presence assay) or quantitative (i.e., which can measure a specific amount of analyte present). Through the use of a cocktail of probe types, the assay can also qualitatively or quantitatively measure the presence of more than one analyte simultaneously. This type of assay can be of commercial importance in clinical and forensic applications, the personal care, pharmaceutical, food and beverage markets, as well as in environmental sample assays.

### FIELD OF THE INVENTION

The invention relates to assays for detecting and determining the presence of specific analytes in a sample. Specifically, the invention relates to the use of sensitized liposomes having adenosine triphosphate (ATP), adenylate kinase (AK) or other luminescence-related compound encapsulated within for detecting and determining the presence of analytes such as bacteria, viruses, genetic material, haptens, immunogenic compounds, chemical compounds and other materials of interest. Liposomes are sensitized through the use of probes which may be oligonucleotides, antibodies or antigens with affinity for the target analyte(s). In addition, the process of specific detection is facilitated by the use of paramagnetic particles.

### BACKGROUND OF THE INVENTION

Through recent innovations in the areas of both instrumentation and reagents, it is now feasible to perform new types of assays that were previously too difficult, too time consuming and/or too costly. Improvements are being made in the performance of assays for the early detection of disease-causing microorganisms in contaminated samples such as clinical samples, personal care and pharmaceutical products, foodstuffs and enviromnental samples, as well as samples for forensic assays.

There are many requirements for methods of screening for specific substances or microorganisms in low levels in specific environments; for example, for the detection of human bacterial pathogens in foods or pharmaceutical products. Public health and quality control groups demand user-friendly detection methods with suitable levels of specificity and sensitivity, but few satisfactory methods exist. Additionally, the medical community and pertinent manufacturers demand detection methods that are robust, reliable, and cost effective, where such methods are simple enough to be performed consistently. For example, in recent years food poisoning has become a major topic of both public and scientific debate. Such contamination has been of great concern to the food producing industries and has led to increased demands for rapid bacterial food screening procedures. These procedures seek to ensure product quality, while allowing timely release for sale. If pathogenic or spoiling bacteria are present in commercially prepared products, then such contamination may occur in low numbers and may be slow-growing. This problem can make conventional bacterial detection a lengthy process, often taking days to complete.

Conventional bacterial detection techniques typically rely upon visual detection of contaminating cells grown on agar plates which is very time consuming and labor intensive. Such conventions need high numbers of bacterial cells (10⁵-10⁸) at the final stage before detection is even possible. These increased cell numbers are usually achieved by laborious and time-consuming procedures involving selective enrichment and isolation steps. Other, more modern detection methods can reduce the need for growth to visually detectable levels, by detecting the chemical components inside, or on the surface of contaminants. Many of these methods are still restricted, however, by finite amounts of the components in the sample, and are therefore still reliant on some degree of cell growth to amplify the amounts of analyte(s) to detectable levels.

The advent of polymerase chain reaction (PCR) techniques that enzymatically amplify selected nucleic acid sequences has had a major impact in many fields where detection and/or analysis of target analytes is performed including in molecular biology and forensics. Despite its benefits, however, there are shortcomings to the technique that have hindered its adoption in other areas where specific detection is desired. For example, because the technique is labor intensive and prone to contamination, it must be performed in a controlled environment and requires a certain level of technical skill on the part of the operator performing the assay. Further, although costs have declined somewhat since its inception, PCR techniques are expensive to perform. These shortcomings have limited the adoption of PCR techniques in industrial microbiology, where a large number of assays must be run every day, frequently in laboratories that are not highly trained, nor properly equipped to handle molecular biology methods.

In the case of inorganic, non-living analytes, such as pesticides, amplification through PCR or enrichment methods is not possible. Most methods have been either time or labor-intensive or require additional, sophisticated equipment.

As an alternative to methods that rely on target amplification, either through growth enrichment or nucleic acid sequence replication, any method that provides signal amplification in an easy-to-use, cost-effective and broadly applicable format will certainly improve the performance, usefulness and value of the assay.

The use of adenosine triphosphate (ATP) as a means to detect microbial contamination has been referenced in the literature as early as 1942 when William McElroy first characterized the connection between ATP and light emission. All living organisms utilize ATP as a source of chemical energy and this ATP can be used in an enzymatic reaction driven by luciferase/luciferin to generate a light signal which can be measured by a luminometer as shown, for example, in U.S. Pat. No. 3,971,703 to Picciollo. The quantity of light generated by such reactions is directly related to the amount of ATP present in the assay. While rapid and easily performed, these reactions are sensitive only to the 10⁻¹² mol/l level, and therefore, typically require a growth enrichment period where an absence/presence test is required. U.S. Pat. No. 5,648,232 to Squirrell shows the use of sequential enzymatic driven reactions such as adenylate kinase (AK) to amplify ATP levels. This protocol can reduce, but usually does not eliminate, the dependency on a growth enrichment period.

The use of liposomes to provide signal amplification has been investigated with limited success. As illustrative examples, the following patents describe diagnostic methods that have been developed to determine the presence of analytes in samples:

U.S. Pat. No. 4,704,355 to Bernstein discloses the use of sensitized liposomes containing ATP which may be used in assays with antibodies and DNA probes. The liposomes of the '355 patent, however, require filtration to isolate bound liposomes and the use of solid microtiter plates which in turn increases costs and is labor intensive. The '355 patent does not employ magnetic particle separation.

U.S. Pat. No. 5,786,151 to Sanders discloses the use of ATP-encapsulation in plastic materials, such as a styrene maleic anhydride copolymer. The encapsulated ATP is intended for use in assays to detect the presence of bacteria or other microorganisms. Since the capsules are prepared from a plastic material, an extremely strong extractant must be used. An example of such an extractant is acetone. The need for a strong extractant renders this product and protocol too difficult to use. For example, acetone is a volatile material and is difficult to use with conventional instrumentation. Further, strong extractants like acetone are detrimental to the luminescent signal generated by the reaction, and negatively impact assay sensitivity. Like Bernstein, Sanders also does not employ magnetic particle separation.

U.S. Pat. Nos. 6,248,596 and 6,086,748 to Durst et al. and Published PCT Application No. WO 03/102541 to Bacumner discloses various uses of fluorescent dye-encapsulated liposomes in a lateral-flow assay for the detection of analytes in a sample. The lateral-flow embodiment of these applications requires the use of a wicking agent and a buffer system, wherein the test components are carried along an assay strip. While convenient, these fluorescence protocols provide some signal amplification, but may not be sufficiently sensitive enough to determine if low levels of analytes are present in a sample.

The development of commercially viable, rapid and specific detection techniques has been addressed world-wide by many companies. Despite these developments, the need remains for a simplified assay protocol that is characterized by sensitive detection and quantitation of analytes in experimental samples. We have discovered that the use of a unique combination of techniques leads to a simplified detection protocol that is more cost-efficient, user-friendly and sensitive. The use of magnetic separation allows for a larger sample volume to be assayed and enables easier and more efficient sample cleanup and target capture, which in turn results in lower background and higher signal, i.e. a greatly improved signal-to-noise ratio. In addition, the inclusion of encapsulated luminescence-related amplificants, such as adenosine triphosphate (ATP) or adenylate kinase, can significantly increase signal generation. Therefore, the sensitivity of such assays is enhanced.

### SUMMARY OF THE INVENTION

The invention relates to methods for detecting an analyte, comprising the steps of obtaining a sample potentially comprising an analyte; providing liposomes comprising a luminescence-related amplificant encapsulated within said liposomes, a buffer and paramagnetic beads; incubating said sample potentially comprising an analyte, said liposomes, and said paramagnetic beads to form a complex of said paramagnetic beads, said analyte, and said liposomes; separating said complex from non-complexed paramagnetic beads and non-complexed liposomes; treating said complex with a liposome extractant to release the contents of said liposomes to form an assay sample; and measuring light via a luminescent means; wherein said liposomes comprise at least one reporter probe; wherein said paramagnetic beads comprise at least one capture probe; and wherein the presence of said analyte is determined by an amount of light emitted from said assay sample.

The invention also relates to a kit for the detection of analytes in a sample comprising a buffer; liposomes, wherein said liposomes comprise an encapsulated luminescence-related amplificant; at least one probe; paramagnetic beads; and a luminescence reagent.

### BRIEF DESCRIPTION OF THE FIGURES

FIG. 1 is a diagram of a nucleic acid based liposome/paramagnetic bead construct.

FIG. 2 is a diagram of an antibody liposome/paramagnetic bead construct.

FIG. 3 depicts a comparison between fluorescent and bioluminescent signals using two types of extractants.

FIG. 4 depicts the correlation between liposome concentration and bioluminescence signal.

FIG. 5 depicts the limit of detection of the liposome/paramagnetic bead assay format compared to a lateral-flow assay format.

### DETAILED DESCRIPTION OF THE INVENTION

The invention relates to the encapsulation of luminescence-related molecules, including, but not limited to, nucleotide triphosphates, such as adenosine triphosphate (ATP), nucleotide diphosphates, such as adenosine diphosphate (ADP), nucleotide monophosphates, such as adenosine monophosphate (AMP), enzymes, such as adenylate kinase (AK), alkaline phosphatase (ALP) and luciferase and associated substrates, such as luminol and luciferin, all of which can be employed to enhance the luminescent detection of microorganisms and compounds in various products and samples. These liposome-encapsulated, luminescence-related compounds can be of commercial importance in clinical and forensic applications, the personal care, pharmaceutical, food and beverage markets, and in environmental sample assays.

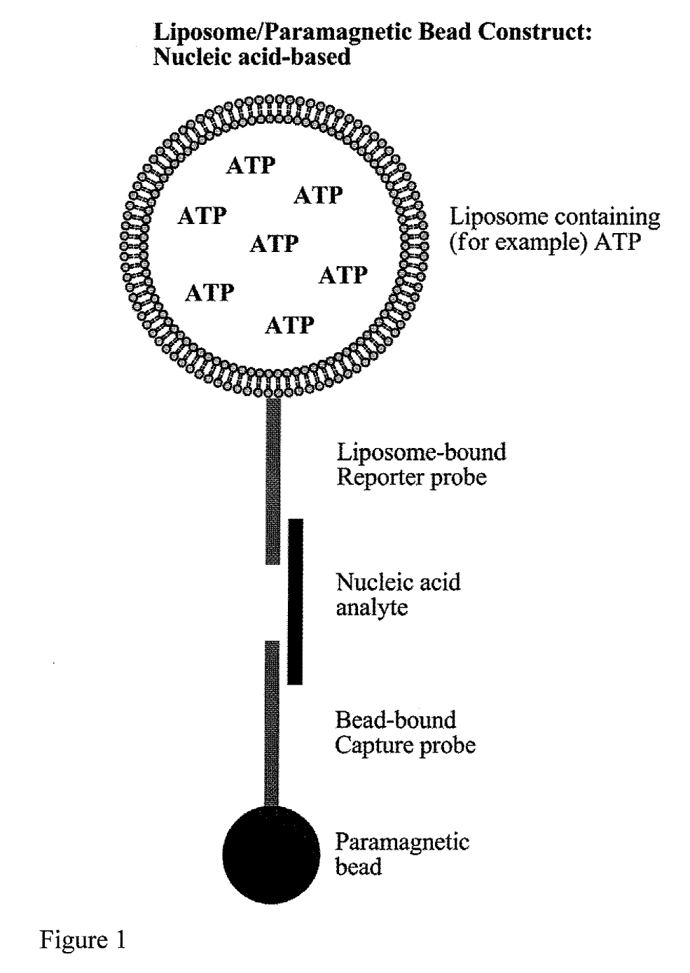

One way to use this technology is for the detection of various analytes. ATP, or other luminescence-related compounds, can be encapsulated within liposomes. These liposomes can bear a probe, an example of which can be an oligonucleotide probe having a specific sequence or structure that in turn can be used to hybridize to a portion of the target analyte. Within the same assay, paramagnetic beads also bear a probe, which in turn can be used to hybridize to a second portion of the target analyte. The probe can be an oligonucleotide probe also having a specific sequence or structure. The result can be a complex of analyte bound to paramagnetic beads and liposomes in a form sometimes referred to as a ‘sandwich’ assay, which is illustrated in FIG. 1. Once the probes hybridize to their targets, the liposomes are washed to remove any non-hybridized probes. Then the hybridized complexes are isolated from the sample using magnetic separation techniques. The complexes are then treated to release the encapsulated ATP, AK, ALP or other luminescence-related compounds from the liposomes. The compounds used to release the liposome contents should have as little adverse effect as possible on the reaction used for the assay of these contents. The released luminescence substrates can then be visualized chemically, for example, in a luciferin/luciferase assay. In addition to being selective, this determination can also be qualitative (i.e., an absence/presence assay) or quantitative (i.e., which can measure a specific amount of analyte present).

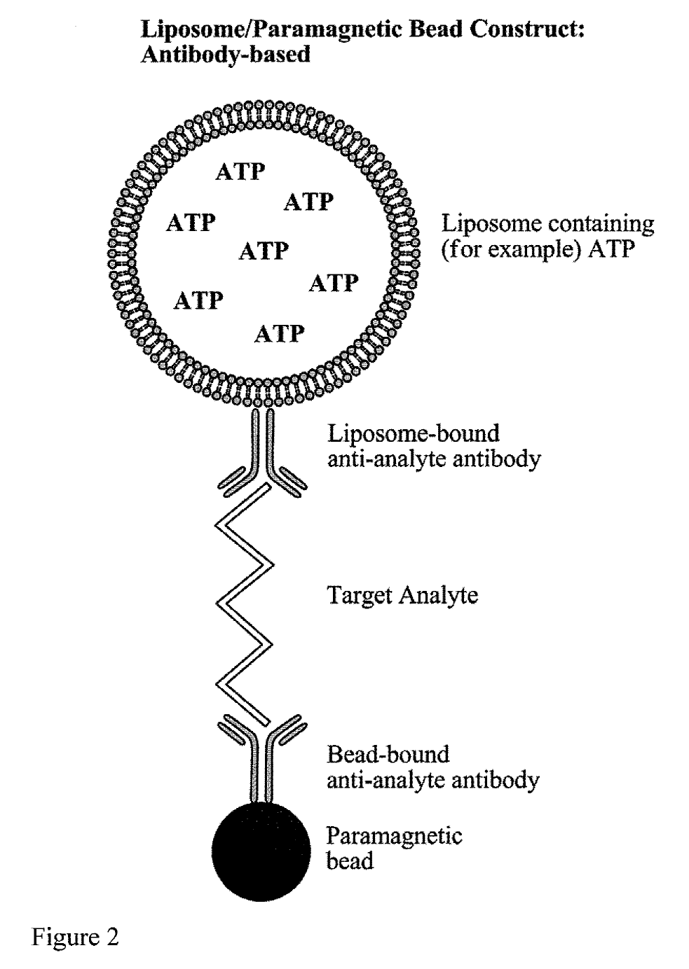

In another example, the probe can be an antibody as illustrated in FIG. 2.

ATP and other luminescence-related compounds can be successfully encapsulated within liposomes, and then the liposomes can be tagged with oligonucleotides, antibodies and/or antigens. These liposomes can be stably maintained over a period of time. The stable liposomes of the invention can be used in an assay for the detection of an analyte or analytes in a sample. These analytes include, but are not limited to, microorganisms (such as viruses, bacteria, and fungi, which may have modified gene sequences), nucleic acid sequences, modified gene sequences, gene products, proteins, antibodies, antigens, haptens, microbial toxins, chemical toxins, molecular markers, immunogenic compounds and chemical compounds (such as pesticides and benzene). The term “molecular markers” includes those compounds, either chemical or biological, that indicate the presence of an organism or compound in a sample. Immunogenic compounds are compounds that can cause an immunological reaction, and include, but are not limited to, allergens, antibodies and antigens.

The analytes, as described, can be found in various types of samples and can be tested by the present method. The sample in which such analytes may be found include, but are not limited to, personal care products, pharmaceutical products, water samples, biological samples, food samples, beverage samples, air samples, nutrient medium samples and clinical samples.

Magnetic separation using paramagnetic beads has been shown to be efficient for the isolation of, for example, cells from blood. Paramagnetic beads can be coated with antibodies or oligonucleotides, and can therefore bind to analytes found in samples. These analytes include, but are not limited to, cells, antigens, nucleic acids, chemical compounds and biological toxins. See Olsvik et al, Magnetic Separation Techniques in Diagnostic Microbiology, 7(1) Clin. Microbiol. Rev. 43-54 (1994) (describing the use of paramagnetic beads in diagnostic assays).

Most particles of this type are known as super-paramagnetic/paramagnetic particles or beads. Such particles or beads can be defined as magnetic when in a magnetic field, but nonmagnetic as soon as the magnetic field is removed, because it would be undesirable to have the particles automatically attach to each other through magnetic forces. One benefit of the use of paramagnetic beads in immuno- and nucleic acid-based assays is that it enables the testing of a much larger sample volume. The use of traditional polymerase chain reaction (PCR) assays is limited because of the small volume of sample that can be tested. For applications such as the testing of foods for bacterial contamination, a much larger amount or volume of sample is often necessary. In addition, PCR sometimes requires large dilutions of samples, which is not necessary for magnetic separation assays.

In one embodiment, the invention relates to methods for detecting an analyte comprising the steps of obtaining a sample potentially comprising a target analyte; providing liposomes comprising a luminescence-related amplificant encapsulated within said liposomes and a probe which is bound to the external surface of said liposomes, a buffer, and paramagnetic beads comprising a probe which is bound to the external surface of said paramagnetic beads; incubating said sample potentially comprising an analyte, said liposomes, and said paramagnetic beads to form a complex formed of the paramagnetic beads, the analyte or portion thereof, and the liposomes; separating paramagnetic bead-bound liposomes from said complex; releasing the contents of said liposomes to form an assay sample; and measuring light via a luminescent means; wherein the presence of said analyte is determined by an amount of light emitted from said assay sample. If the analyte is a nucleic acid, then it can be either RNA or DNA.

In another embodiment of the invention, the presence of an analyte can be determined qualitatively or quantitatively. Either the presence of the analyte in a sample can be read as either positive or negative, or as a result that can be proportional to the sample concentration of the analyte in the sample. The presence of a specific amount of the analyte can be determined via the luminescence reaction. The luminescence is typically a result of the presence of a luminescence-related amplificant contained within, and released from, the liposome. The luminescence-related amplificant can be, but is not limited to, adenosine triphosphate (ATP), adenylate kinase (AK), alkaline phosphatase (ALP), luminol or luciferase/luciferin cocktail.

In another embodiment of the invention, if the analyte is, for example, a specific RNA or DNA sequence within a microorganism, than the analyte can be extracted by use of one of a variety of methods known to one skilled in the art to prepare the sample for the hybridization/coupling step of the assay. The liposome extractant can be effective at releasing the contents of the liposomes and/or microorganisms, and can have a limited effect, or no negative effect, on the luminescent reaction. In other words, the liposome extractant does not adversely affect subsequent detection reactions. The extractant can comprise surface-active gluconate compounds or derivatives and ethylene-amine compounds or derivatives. Extractant 2 is such a material. The contents of the liposomes and/or microorganisms can be released by lysis via the extractant. Alternatively, the contents of the liposomes and/or microorganisms can be released by the creation of pores in the membrane of the liposomes and/or microorganisms.

In another embodiment of the invention, the paramagnetic beads used in the present method are labeled and can be comprised of a capture probe, antibody or antigen. The labels can hybridize to each other and ultimately bind to the analyte. The labels of the paramagnetic beads and probes can be joined by binding methods familiar to those skilled in the art, including but not limited to, biotin and streptavidin.

In another embodiment of the invention, the liposomes used in the present method are labeled and can be comprised of a reporter probe, antibody or antigen. The labels are such that they will hybridize to each other and ultimately bind to the analyte.

In order to separate the paramagnetic beads bound to the liposomes, a magnetic device can be used. In one embodiment of the use of such a device, the paramagnetic beads can be attracted to the device, enabling the beads to be separated from the initial incubation solution and assayed for the presence of a bound analyte. Examples of such devices include, but are not limited to, devices that can be placed into the sample to separate bound paramagnetic complexes from an unbound sample, such as a PickPen™ (Bio-Nobile Oy, Turku, Finland), or devices that immobilize paramagnetic substances allowing for an unbound sample to be removed from bound paramagnetic complexes, such as magnetic tube racks.

The assay itself can be performed using combinations of luminescence-related amplificant and luminescence reagents. For example, when said luminescence-related amplificant is adenylate kinase (AK) (or similar), the luminescence assay can be performed using luciferase, luciferin and adenosine diphosphate (ADP) (or similar). When said luminescence-related amplificant is adenosine triphosphate (ATP), the luminescence assay can be performed using luciferase and luciferin. When said luminescence-related amplificant is a luciferase/luciferin, the luminescence assay can be performed using ATP. When said luminescence-related amplificant is luminol, the luminescence assay can be performed using hydrogen peroxide. When said luminescence-related amplificant is alkaline phosphatase (ALP), the luminescence assay can be performed using a suitable substrate. Alkaline phosphatase assays can use a wide variety of substrates, such as 1,2-dioxetane.

In a further embodiment, the invention further relates to a kit for the detection of such analytes in a sample comprising a buffer; liposomes, wherein said liposomes comprise an encapsulated luminescence-related amplificant; at least one probe or antibody; paramagnetic beads; and a luminescence reagent. The encapsulated luminescence-related amplificant can be, but is not limited to, adenosine triphosphate (ATP), adenylate kinase (AK), luminol, alkaline phosphatase (ALP) or a luciferase/luciferin cocktail. The liposomes of the kit can be labeled with at least one reporter probe. The liposomes can also be labeled with a cocktail of different probes, enabling the simultaneous detection of more than one analyte if desired. For example, if one desires to test the presence of several pesticides that have been applied together, such as organochlorine-based pesticides (such as Heptaclor) and/or organophosphorous-based pesticides (such as Malathion), the use of a cocktail of probes enables such detection. At least one reporter probe can be specific for a target nucleic acid sequence, antibody, hapten or antigen.

The paramagnetic beads of the kit can be labeled and be further comprised of a capture probe, which can also be labeled. The labels are such that the capture probe can bind to the paramagnetic beads, and ultimately bind to the probe on the liposomes. The labels of the paramagnetic beads and capture probes can be, but are not limited to, biotin and streptavidin. The paramagnetic beads can be pre-labeled or the assay can include a labeling step.

In a further embodiment of the invention, the kit can be comprised of a device or means for magnetic capture. The paramagnetic beads can be attracted to the device, enabling the beads to be separated from the initial incubation solution and assayed for the presence of a bound analyte. Examples of such devices include, but are not limited to magnetic picks and magnetic test tube racks. The kit can be comprised of further a positive and negative control, so that the assay samples can be read and compared with said controls. Written instructions for use of the kit can also be included.

As used herein, the term “amplificant” describes a compound or compounds that function as a means for detection in a luminescence assay. By use of such amplificants, the presence of an analyte can be determined with enhanced sensitivity. The use of such a compound or compounds can cause a readable signal to be generated via luminescence. Compounds that can be considered to be amplificants include, but are not limited to, adenosine triphosphate, adenylate kinase, adenosine diphosphate, luminol, luciferin, luciferase and/or the alkaline phosphatase family of enzymes.

It is well known that liposomes can be used in assays for the determination of the presence of various organisms and compounds. Liposomes for use in assays can be prepared by any method known to persons skilled in the art. Methods of producing liposomes for use in assays, as well as assay methods, are disclosed in the following references: Mason et al., A Liposome-PCR Assay for the Ultrasensitive Detection of Biological Toxins, 24(5) Nature Biotechnology 555-557 (2006); Edwards et al., Flow-Injection Liposome Immunoanalysis (FILIA) with Electrochemical Detection, 7(9) Electroanalysis 838-845 (1995); Yamamoto et al., Automated Homogenous Liposome-Based Assay System for Total Complement Activity, 41(4) Clin. Chem. 586-590 (1995); Frost et al., A Novel Colourimetric Homogenous Liposomal Immunoassay Using Sulphorodamine B, 4(3) Journal of Liposome Research 1159-1182 (1994); Kim et al, Liposome Immunoassay for Gentamicin Using Phospholipase C, 170 Journal of Immunological Methods 225-231 (1994); Haga et al., An Improved Chemiluminescence-Based Liposome Immunoassay Involving Apoenzyme, 38 Chem. Pharm. Bull. 252-254 (1990); Gerber et al, Liposome Immunoassay for Rapid Identification of Streptococci from Throat Swabs, Journal of Clinical Microbiol. 1463-1464 (1990); Nakamura et al., A Liposome Immunoassay Based on a Chemiluminescence Reaction, 37(6) Chem. Pharm. Bull. 1629-1631 (1989); Monroe, Liposome Immunoassay, Immunoassay Technology, Vol. 2. (1986); Alving et al, Preparation and Use of Liposomes in Immunological Studies, Liposome Technology, Vol. 3, Chapter 21 (1986).

The liposomes of the invention can have a diameter of between about 150 μm and about 400 μm. Further, the diameter of the liposomes can be about 150 μm, about 200 μm, about 250 μm, about 300 μm or about 350 μm. Generally, the diameter of the liposomes can be varied within this range. The specific diameter can be selected in order to vary the assay sensitivity. Further, the liposomes can be of multilamellar and/or unilamellar or a combination of both with compartmentation containing separated components or amplificants of the detection system. Increasing the sensitivity of the assay in this way can be important in situations where the analyte is present in a limited amount. Conversely, decreasing the sensitivity by reducing the liposome diameter can be of benefit when analyte is present in non-limiting amounts. Smaller liposomes can be preferable, for example, for reasons of stability.

The liposomes of the invention can have a range in the concentration of amplificant encapsulated within each liposome. The concentration of adenosine triphosphate or other luminescent-related components contained in the liposomes can be from 10⁻¹⁸ to 1 M/L. The concentration of APT can be about 150 nM. The specific concentration can be chosen in order to alter the assay sensitivity. Increasing the sensitivity of the assay can be important in situations where the analyte is present in a limited amount. Decreasing the sensitivity of the assay in situations where the analyte is non-limiting can result in cost savings.

The liposomes of the invention can be labeled with at least one reporter probe. The probe can also be a cocktail of different probes. Furthermore, the at least one reporter probe can be specific for a target. The target can be, but is not limited to, a nucleic acid sequence, an antibody, an antigen, a hapten or a chemical entity. The use of probes specific for targets on both the paramagnetic beads and the liposomes can enable the preferential binding of the liposomes, target and paramagnetic beads, resulting in paramagnetic bead bound liposomes in a sandwich formation with the target. Thus, the presence of an analyte will result in a “sandwich” that can be detected via the luminescence assay of the present invention.

The probes, as either nucleic acids, antibodies or antigens, are generally components of a “coupling group.” Such a group is any group of two or more members, where each of which are capable of recognizing a particular chemical, spatial or polar organization of a molecule, e.g., an epitope or determinant site. Suitable coupling groups in accordance with the invention include, but are not limited to, antibody-antigen, receptor-ligand, biotin-streptavidin, sugar-lectins and complementary oligonucleotides of RNA, DNA or PNA (peptide nucleic acid). For example, an antibody, sufficiently different in structure from the analyte of interest, can be employed as a member of a coupling group with a suitably derivatized binding material (i.e., derivatized with the specific antigen of the antibody). Illustrative members of the coupling groups include, but are not limited to, avidin, streptavidin, biotin, anti-biotin, anti-fluorescein, fluorescein, anti-digoxin, digoxin, anti-dinitrophenyl (DNP), DNP, generic oligonucleotides (e.g., substantially dC and dG oligonucleotides), antibodies and antigens. As an example, in one embodiment of the invention, biotin functions as one member of a coupling group for liposomes or any membrane comprising streptavidin or an anti-biotin antibody.

The probes attached to the liposomes are termed “reporter” probes, and are specific for the analyte or a portion thereof. The probes attached to the paramagnetic beads are termed “capture” probes. These probes are also specific for the analyte or a portion thereof. When the “reporter” and “capture” probes bind to or couple with the analyte or portion thereof, a complex is formed between the paramagnetic beads, the analyte and the liposomes. This complex is commonly referred to as a “sandwich.” This “sandwich” is formed from these three components, which can then be separated from the non-bound, or non-complexed, components. The presence or absence of the analyte, and/or the amount thereof can then be determined by luminescence methods.

The probes can be attached to the liposomes and paramagnetic beads by conventional methods known to those skilled in the art. To illustrate such methods, the following references teach several non-limiting examples: Torchilin et al., p-Nitrophenylcarbonyl-PEG-PE-Liposomes: Fast and Simple Attachment of Specific Ligands, Including Monoclonal Antibodies, to Distal Ends of PEG Chains Via p-Nitrophenylcarbonyl Groups, 1511(2) Biochim. Biophys. Acta 397-411 (2001); Velev, Assembly of Protein Structures on Liposomes by Non-Specific and Specific Interactions, 34 Adv. Biophys. 139-157 (1997); Corley et al, Binding of Biotinated-Liposomes to Streptavidin is Influenced by Liposome Concentration, 1195(1) Biochim. Biophys. Acta 149-156 (1994); Loughrey et al, Characterisation of Biotinylated Liposomes for In Vivo Targeting Applications, 332(1-2) FEBS Lett. 183-188 (1993); Loughrey et al., Optimized Procedures for the Coupling of Proteins to Liposomes, 132(1) J. Immunol. Meth. 25-35 (1990); Loughrey et al, A Non-Covalent Method of Attaching Antibodies to Lipsomes, 901(1) Biochim. Biophys. Acta 157-160 (1987); Chiruvolu et al., Higher Order Self-Assembly of Vesicles by Site-Specific Binding, 264(5166) Science 1753-1756 (1987); and Published PCT Application No. WO 03/102541.

Bioluminescence has been commonly used for the detection of microorganisms. For example, the firefly luciferase assay of ATP uses the emission of light in the luciferase catalyzed reaction between luciferin and ATP. Bioluminescence compounds and uses thereof have been described in several publications and patents. For example, U.S. Pat. Nos. 6,949,351; 6,720,192; 6,200,767; 5,837,465; 5,798,214; 5,700,645; 5,648,232; 3,971,703; and 3,933,592. In addition, the following references disclose subject matter relating to bioluminescent compounds and the use thereof. Klegerman, Quantitative ATP Analysis Automated Microbial Identification and Quantitation. pp 259-273 (Buffalo Grove, Ill. Interpharm Press, Inc. 1996); Lundin, ATP Assays in Routine Microbiology: From Visions to Realities in the 1980s, ATP Luminescence, eds. Stanley, P. E., McCarthy, B. J. & Smither, R., pp 11-31, (Oxford: Blackwell Scientific Publications 1989); Stanley, A Concise Beginner's Guide to Rapid Microbiology Using Adenosine Triphosphate (ATP) and Luminescence. ATP Luminescence, eds. Stanley, P. E., McCarthy, B. J. & Smither, R., pp 1-11, (Oxford: Blackwell Scientific Publications 1989); McElroy et al., Firefly and Bacterial Luminescence: Basic Science and Applications, 5 Journal of Applied Biochemistry 197-209 (1983); Campbell, Living Light: Biochemistry, Function and Biomedical Applications, 24 Essays in Biochemistry 41-81 (1989); and Sala-Newby et al., A Concise Beginner's Guide to Rapid Microbiology Using Adenosine Triphosphate (ATP) and Luminescence, ATP Luminescence, eds. Stanley, P. E., McCarthy, B. J. & Smither, R., pp 1-11. (Oxford: Blackwell Scientific Publications 1989).

Paramagnetic beads have been used in enzyme immunoassay systems and also fluorescent assays. Antibodies, bacteria, proteins and genetic material can be detected using paramagnetic bead immuno- and fluorescent assays. Such techniques are disclosed in the following illustrative references: Katie A. Edwards and Antje J. Baeumner, Liposomes in Analyses, 68 Talanta 1421-1431 (2006); Katie A. Edwards and Antje J. Baeumner, Analysis of Liposomes, 68 Talanta 1432-1441 (2006); Safarik et al, Magnetic Techniques for the Isolation and Purification of Proteins and Peptides, 2 Biomagnetic Research and Technology 7 (2004); Saiyed et al, Application of Magnetic Techniques in the Field of Drug Discovery and Biomedicine, 1(1) Biomagnetic Research and Technology 2 (2003); Matsunaga et al., Chemiluminescence Enzyme Immunoassay Using Bacterial Magnetic Particles, 68(20) Anal. Chem. 3551-3554 (1996); and Nakamura et al., Detection and Removal of Escherichia coli Using Fluorescein Isothiocyanate Conjugated Monoclonal Antibody Immobilized on Bacterial Magnetic Particles, 65(15) Anal. Chem. 2036-2039 (1993).

The present invention is described in further detail in the following non-limiting examples. The great variety of options falling within the scope of the invention will be readily determinable by those skilled in the art upon consideration of the general method described above and exemplified below.

### EXAMPLE 1

### Encapsulation of ATP Into Liposomes

A total of six batches of liposomes were produced, in which ATP was encapsulated at four different concentrations. Liposomes containing 0 mM (one batch); 150 mM (three batches); 300 mM (one batch); and 400 mM (one batch) were prepared. It was found that, at ATP concentrations of 300 mM and 400 mM, significant aggregation of the liposomes occurred. Liposomes carrying 150 mM ATP remained unaggregated, and were suitable for use in the detection assays of the invention.

Generally, liposomes can be prepared by any method known to one skilled in the art. Several methods for encapsulating ATP and other luminescence-related amplificants are known. For example, Guo-Xing described and evaluated four methods for the encapsulation of ATP. (Guo-Xing et al, Adenosine Triphosphate Liposomes: Encapsulation and Distribution Studies, 7(5) Pharm. Res. 553-557 (1990)). Specifically, Guo-Xing described thin film-formed vesicles, reverse-phase evaporation vesicles, double emulsification vesicles and improved emulsification vesicles, and methods of making thereof. Liang et al. also described ATP-encapsulated liposomes. (Liang et al., Encapsulation of ATP into Liposomes by Different Methods: Optimization of the Procedure 21(3) J. Microencapsulation 251-261 (2004)). Liang also disclosed the reverse-phase evaporation, as well as thin lipid film hydration, pH gradient and freeze-thawing methods of preparing ATP-encapsulated liposomes.

The liposomes in Example 1 were produced by reverse-phase evaporation and extruded through 2 μm and 0.4 μm filters in order to control the size of the liposome. The liposomes were purified by gel filtration using a Sephadex™ G50 column. The osmolality of the encapsulant solution was adjusted, when necessary, to ensure the integrity of the liposomes.

After gel filtration, fractions of different optical densities, termed “high,” “medium,” “low,” and “lowest” were collected and pooled. Liposomes were characterized in terms of particle size and encapsulation efficiency, as described below:

### Particle Size.

The vesicle diameter was determined by dynamic light scattering, and the data is shown in Table I. Except for Batch No. 3, all liposome preparations were sequentially extruded through 2- and 0.4-μm filters.

TABLE I
Liposome Diameters Obtained from Dynamic Light Scattering
Measurements.

| | | | | Number of |
|---|---|---|---|---|
| Batch | Cone. ATP | Liposome | Std. | total lipids |
| number | encapsulated (mM) | diameter, nm | Deviation | per liposome |
| 1 | 150 | 386 | 10 | 2,243,261 |
| 2 | | 375 | 0.6 | 2,104,909 |
| 3 | | 338* | 8 | 1,706,044 |
| 4 | 0 | 314 | 3 | 1,479,202 |
| 5 | 300 | — | — | — |
| 6 | 400 | 452 | 12 | — |

*This batch was sequentially extruded through 1- and 0.4-μm filters

From the above data, one can see when under the same conditions, there is successful reproducibility (Batch Nos. 1 and 2) with respect to the hydrodynamic diameter. It was assumed that the absence of the encapsulant molecules in empty liposomes was responsible for the smaller diameters. The aggregated 400 mM ATP liposomes displayed a larger diameter which was, in part, due to liposome aggregation. The 300 mM ATP liposomes were not analyzed.

The number of total lipids per liposome was calculated by assuming a lipid bilayer thickness of 4 μm and using the equation for surface area of sphere and typical values for the areas occupied by one lipid (0.52 nm² for DPPC, 0.45 nm² for DPPG, and 0.30 nm² for cholesterol).

### Encapsulation Efficiency.

Encapsulation efficiency (EE) was determined by assaying ATP-encapsulated liposomes (high fraction, Batch No. 3) in Bartlett assay without the lipid extraction. Total phosphorous content obtained from the calibration curve included phosphorous from phospholipids and ATP and was equal to 23.22 nmol/μL. The ATP concentration was found by subtracting phospholipid phosphorus from total phosphorus content and by dividing the result by 3 (3 phosphorous atoms per ATP molecule) and was equal to 5.12 nmol/μL, or 1⁻¹⁸ mol of ATP/liposome. The inner volume of one liposome was equal to 1.88⁻¹⁷ L; therefore, the molar concentration of ATP inside the liposome was equal to 53 mM, which comprised the EE of 35%. Therefore, each liposome contained as many as 602,500 molecules of ATP, which was well within an expected range of desirable liposomes.

### EXAMPLE 2

### Performance of ATP-Encapsulated Liposomes

Liposomes encapsulating 150 mM ATP were used for this example. In order to estimate the potential improvement in assay sensitivity possible by encapsulation of ATP, we compared the ability to detect liposomes encapsulating either 150 mM ATP, a bioluminescence-related amplificant, or 150 mM sulforhodamine B (SRB), fluorescence-related amplificant. Each set of liposomes was subjected to serial 10-fold dilutions and analyzed by using the assay shown in Table II to determine which population of liposomes could be detected at the highest possible dilution.

Both sets of liposomes were treated with either 60 mM n-octylglucopyranoside (OG, ‘Extractant 1’) or ‘Extractant 2’ in order to release the amplificant from the liposomes. It was found that the SRB-encapsulated liposomes could be detected at dilutions of 1/100,000 whether intact, or disrupted via Extractant 1 or Extractant 2 (Table III). The ATP-encapsulated liposomes, on the other hand, could be detected at dilutions as high as 1/100 million when disrupted with Extractant 2 (Table IV). Based on these findings, we can expect at least a 1000-fold more sensitive assay when ATP-encapsulated liposomes are used in place of SRB-encapsulated liposomes.

TABLE II
Assay Configuration.

| Assay component | Volume (μL) |
|---|---|
| Liposomes | 10 |
| Extractant 1 or Extractant 2 | 200 |
| Luciferin/Luciferase (for ATP-assays) | 100 |

TABLE III
Serial Dilution of Liposomes Encapsulating 150 mM Sulforhodamine
B (SRB) Using Extractant 1 or Extractant 2 (Data in Fluorescence Units).

| | Intact SRB | Extracted | Extracted |
|---|---|---|---|
| | Liposomes, | Liposomes, | Liposomes, |
| Sample | ‘background’ | Extractant 1 | Extractant 2 |
| No liposomes, buffer | 3 | 24 | 2 |
| 1:100,000,000 | 2 | 24 | 3 |
| 1:10,000,000 | 3 | 32 | 6 |
| 1:1,000,000 | 5 | 32 | 11 |
| 1:100,000 | 20 | 104 | 63 |
| 1:10,000 | 153 | 753 | 718 |
| 1:1,000 | 1211 | 7121 | 5898 |
| 1:100 | 6150 | 71536 | 51852 |
| 1:10 | 7009 | Overload | Overload |

TABLE IV
Serial Dilution of Liposomes Encapsulating 150 mM ATP using
Extractant 1 or Extractant 2 (Data in Relative Light Units).

| | Intact ATP | Extracted | Extracted |
|---|---|---|---|
| | Liposomes, | Liposomes, | Liposomes, |
| Sample | ‘background’ | Extractant 1 | Extractant 2 |
| No liposomes, buffer | 55 | 9 | 500 |
| 1:100,000,000 | 56 | 22 | 1,588 |
| 1:10,000,000 | 62 | 158 | 11,094 |
| 1:1,000,000 | 110 | 1,483 | 110,251 |
| 1:100,000 | 623 | 14,557 | 1,053,620 |
| 1:10,000 | 4,563 | 155,404 | Overload |
| 1:1,000 | 29,058 | Overload | Overload |
| Undiluted | Overload | — | Overload |
| | (99,999,999) | | |

TABLE V
Signal-to-Noise Ratios: Comparison of Table III and Table IV.

| | Fluorescence, | Bioluminescence, | Bioluminescence, |
|---|---|---|---|
| Sample | Extractant 1 | Extractant 1 | Extractant 2 |
| No liposomes, | 8 | 0 | 9 |
| buffer | | | |
| 1:100,000,000 | 12 | 0 | 28 |
| 1:10,000,000 | 11 | 3 | 179 |
| 1:1,000,000 | 6 | 13 | 1002 |
| 1:100,000 | 5 | 23 | 1691 |
| 1:10,000 | 5 | 34 | N/A |
| 1:1,000 | 6 | N/A | N/A |
| 1:100 | 12 | N/A | N/A |
| 1:10 | N/A | N/A | N/A |

In order to compare the dual effects observed from using ATP in combination with the Extractant 2, the data from Tables III and IV was compared. This comparison was done by dividing the experimental signal obtained by the signal obtained using intact liposomes for each liposome dilution. The results are shown in Table V.

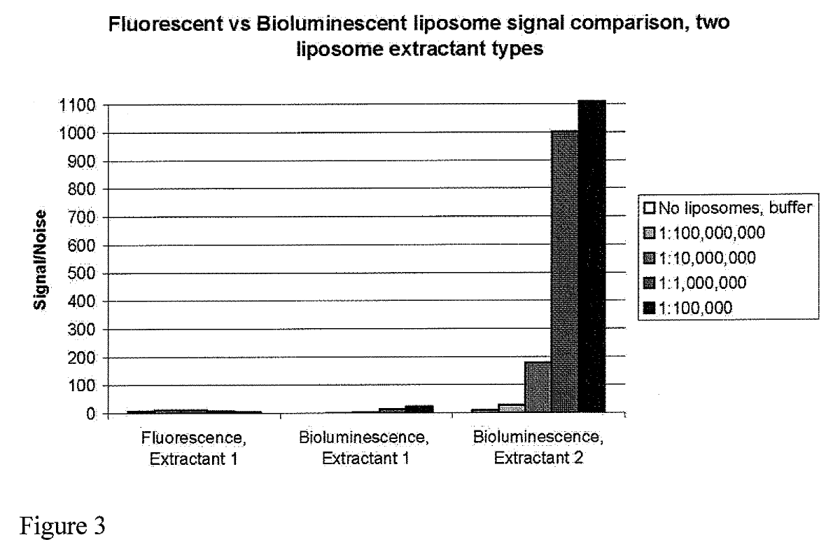

It was noted that the signals obtained using Extractant 2 were approximately 100 times higher than those obtained when liposomes were disrupted with Extractant 1, as noted in FIG. 3. Although the effect was not further investigated, it was assumed that the superior performance of Extractant 2 was due to its composition, which had been previously optimized to minimize any negative effects on either the luciferase or luciferin required in the assay. The use of non-optimal Extractant 1, however, was likely to have caused either the denaturation of the luciferase enzyme and/or its substrate (luciferin) or interfered with some other aspect of the bioluminescence assay. Therefore, since Extractant 2 can effectively disrupt the ATP-encapsulated liposomes in a manner that was not deleterious to the assay format, Extractant 2 was employed for all experimental protocols.

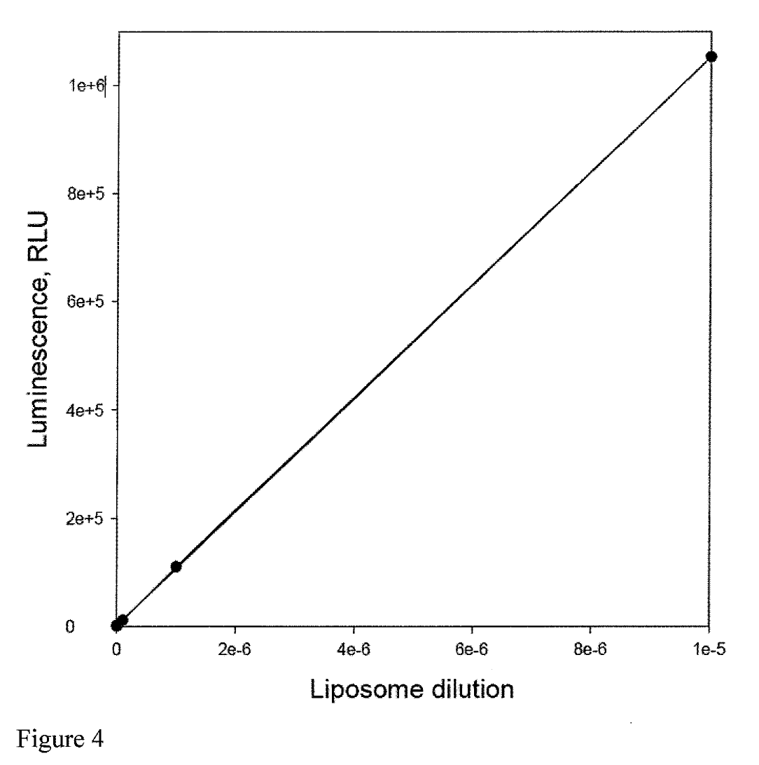

As expected, a direct linear relationship exists between the concentration of diluted liposomes and the bioluminescence signal generated with a correlation coefficient of 0.9999, as shown in FIG. 4. This correlation was significant since the assay configuration could be based upon a direct linear relationship between the concentration of the target nucleic acid sequence and the amount of liposomes introduced into a luminometer, such as the Celsis Advance™ instrument. Therefore, it was anticipated that a direct linear relationship could exist between the concentration of the target analyte and the amount of detectable bioluminescence, which indicated that assays capable of quantitating analyte levels were possible.

### EXAMPLE 3

### Demonstration of Further Improvement in Performance Through the Use of Paramagnetic Beads

### Immobilization of Oligonucleotides on Paramagnetic Bead Surface.

Biotinylated capture oligonucleotide probes were conjugated by methods known to those skilled in the art to streptavidin coated paramagnetic beads. After conjugation and washing, the labeled paramagnetic beads were stored at 4° C. for several months. It was noted that, while this particular example relates to the immobilization of oligonucleotides, antibodies or other compounds capable of binding to a sample can be also be used.

### Determination of Detection Limits

The limits of detection of the assay system of the present invention were determined using a synthetic nucleotide sequence. A sandwich hybridization assay was performed in microplate wells using 15 μl of a 1:10 dilution of liposomes and 5 μl (1.25 μg) of paramagnetic beads. The bead-target-liposome complex was washed using two types of magnetic devices to retain the complex, resuspended in buffer and then transferred to test tubes for bioluminescence recordation. The device that showed the best results was the PickPen™ (Bio-Nobile Oy, Turku, Finland). This device enabled one to remove the paramagnetic beads from the assay and wash buffers. Because the beads were actually removed from the solution, a more thorough washing was performed, and thus reduced any background noise and/or contamination.

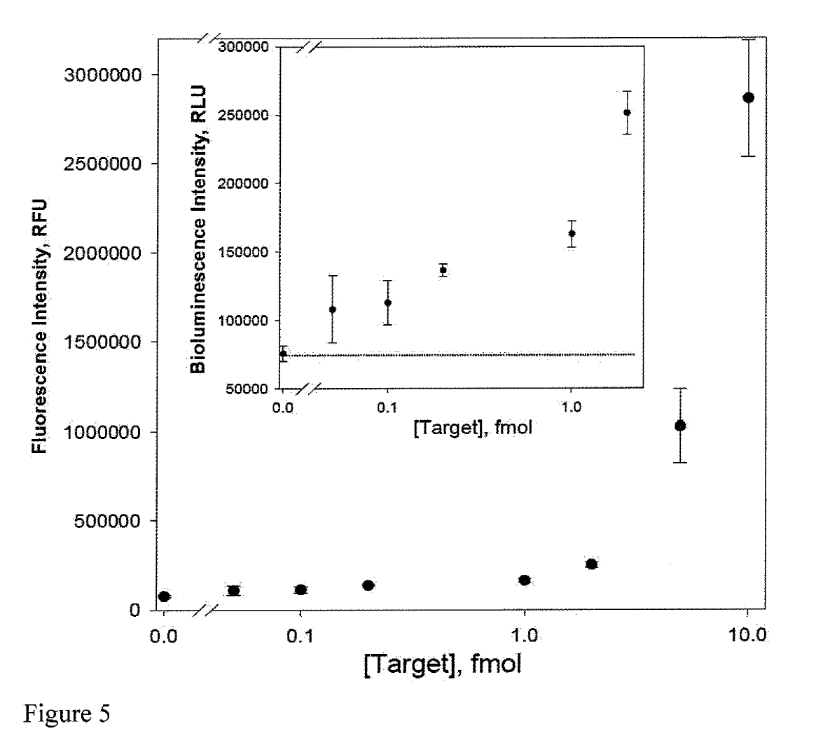

As noted in FIG. 5, the current minimum limit of detection was determined to be 0.1 fmole of target/well (assumed “cut off” value equal to the bioluminescence value representing the mean, plus three standard deviations). As compared to the limit of detection (LoD) of other assay formats, e.g., a fluorescent-based liposome assay format, this LoD was at least 100-times more sensitive.

### EXAMPLE 4

### Detection of RNA from E. coli

A sample containing E. coli 0157:H7: 9 was evaluated in order to determine if the assay method was effective. The method was evaluated using targets for rRNA and mRNA using the following method: The sample containing E. coli 0157:H7: 9 cells was subjected to a brief heatshock in order to induce the synthesis of clpB (heatshock) mRNA. Total RNA including mRNA and rRNA was extracted from the E. coli using the commercially available RNEasy® kit from Qiagen (Qiagen Inc, Valencia, Calif., USA). The hybridization step was performed by combining 30 μl buffer; 5 μl of reporter probe at 200 fmol/μl; 5 μl of a 1:10 dilution of liposomes encapsulating ATP; and 5 μl of extracted RNA. The mixture was incubated for 20 minutes at 41° C. 5 μl of paramagnetic beads (1.2 μg) that had been labeled with capture probe were added and incubated for an additional 30 minutes at room temperature. Bound liposomes were washed and removed from the sample using magnetic means and resuspended in wash buffer. The washing step was performed three times. The washed bead/liposome complex was resuspended in 1× HEPES-saline buffer at pH 7.5, transferred into assay tubes, and then placed into a luminometer. The luminometer was programmed to inject extractant into the assay tube to release the ATP from the liposomes. Following extraction, the luminometer injected bioluminescence reagents and the resulting bioluminescent light output was read and recorded.

Three types of RNA were measured in this Example. These types were clpB mRNA, 16S rRNA and 23S rRNA (the latter two being ribosomal RNA), all of which yielded positive signals with the 23S rRNA yielding the most pronounced bioluminescence signals. Results (given in relative light units (RLU)) are presented in Table VI below.

TABLE VI
Detection of RNA from E. coli 0157:H7: 9 Using Bioluminescence.

| | | Bioluminescence (RLU) +/− | Signal/ |
|---|---|---|---|
| RNA Type | Concentration | Standard Deviation | Noise |
| clpB mRNA | Control | 24,200 +/− 500 | 2 |
| | Test | 55,600 +/− 1,700 | |
| 16S rRNA | Control | 21,600 +/− 0 | 4 |
| | Test | 79,900 +/− 800 | |
| 23S rRNA | Control | 28,400 +/− 900 | 61 |
| | Test | 1,739,000 +/− 104,300 | |

### PROPHETIC EXAMPLE 1

A sample containing an unknown bacterial contamination can be evaluated by the assay of the present invention. Total bacterial RNA including mRNA and rRNA can be extracted from the sample using one of a variety of methods known to those skilled in the art. Next, the hybridization step can be performed by combining 30 μl buffer; 5 μl of reporter probe at 200 fmol/μl; 5 μl of a 1:10 dilution of liposomes encapsulating ATP; and 5 μl of extracted RNA. The mixture can then be incubated for 20 minutes at 41° C. 5 μl of capture-probe labeled paramagnetic beads (1.2 μg) can next be added to the sample and incubated for an additional 30 minutes at room temperature. Any bound liposomes can be washed by removal from the sample using a magnetic means (e.g., a PickPen™ (Bio-Nobile Oy, Turku, Finland)) and resuspended in wash buffer. The washing step should be performed at least three times. The washed bead/liposome complex can then be resuspended in an assay buffer, transferred into assay tubes, and placed into a luminometer. The luminometer should be programmed to inject extractant into the assay tube so as to release the ATP from the liposomes. Following extraction, the luminometer can inject bioluminescence reagents to the sample. The resulting bioluminescent light output can then be read and recorded.

While various embodiments of the present invention have been described above, it should be understood that such disclosures have been presented by way of example only, and are not limiting. Thus, the breadth and scope of the present invention should not be limited by any of the above-described exemplary embodiments, but should be defined only in accordance with the following claims and their equivalents.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference in their entirety.

We claim:

1. A method for detecting an analyte comprising the steps of:
a) obtaining a sample potentially comprising an analyte;
b) providing liposomes comprising a luminescence-related amplificant encapsulated within said liposomes, a buffer and paramagnetic beads;
c) incubating said sample potentially comprising an analyte, said liposomes, and said paramagnetic beads to provide a complex of said paramagnetic beads, said analyte and said liposomes;
d) separating said complex from non-complexed paramagnetic beads and non-complexed liposomes;
e) treating said complex with a liposome extractant to release the contents of said liposomes to form an assay sample; and
f) measuring light via a luminescent means;
wherein said liposomes of step b) comprise at least one reporter probe; wherein said paramagnetic beads of step b) comprise at least one capture probe, and wherein the presence of said analyte is determined by an amount of light emitted from said assay sample.

2. The method of claim 1, wherein the presence of said analyte can be determined qualitatively or quantitatively.

3. The method of claim 1, wherein said luminescence-related amplificant is selected from the group consisting of adenosine triphosphate (ATP), adenylate kinase (AK), luminol, alkaline phosphatase (ALP) and a luciferase/luciferin cocktail.

4. The method of claim 3, wherein, when said luminescence-related amplificant is AK, said luminescence assay is performed using luciferase, luciferin and adenosine diphosphate (ADP).

5. The method of claim 3, wherein, when said luminescence-related amplificant is ATP, said luminescence assay is performed using luciferase and luciferin.

6. The method of claim 3, wherein, when said luminescence-related amplificant is a luciferase/luciferin, said luminescence assay is performed using ATP.

7. The method of claim 1, wherein the analyte is selected from the group consisting of bacteria, fungi, viruses, modified gene sequences, gene products, immunogenic compounds and molecular markers.

8. The method of claim 7, wherein the analyte comprises RNA, DNA, an antibody or an antigen.

9. The method of claim 8, wherein said RNA, said DNA or said antigen is isolated by removal from said analyte with a liposome extractant solution.

10. The method of claim 1, wherein the paramagnetic bead bound liposomes are washed with a wash buffer prior to removal of the contents of said liposomes.

11. The method of claim 1, wherein the separating of step d) is performed using a device comprising means for paramagnetic capture.

12. The method of claim 1, wherein the liposome extractant comprises a surface-active gluconate compound or derivative and an ethylene-amine compound or derivative.

13. The method of claim 12, wherein the liposome extractant does not chemically and/or adversely affect subsequent detection reactions.

14. The method of claim 1, wherein said paramagnetic beads and said capture probe are labeled.

15. The method of claim 14, wherein said paramagnetic beads are labeled with biotin and said capture probe is labeled with streptavidin.

16. The method of claim 14, wherein said paramagnetic beads are labeled with streptavidin and said capture probe is labeled with biotin.

17. The method of claim 1, wherein said sample is selected from the group consisting of a water sample, a biological sample, a food sample, a beverage sample, an air sample, a nutrient medium sample and a clinical sample.

18. The method of claim 1 wherein the liposomes have a diameter of between about 150 microns and about 400 microns.

19. The method of claim 18, wherein the diameter of said liposomes is selected to vary assay sensitivity.

20. The method of claim 1, wherein said liposomes are unilamellar or multilamellar.

21. The method of claim 1, wherein said at least one reporter probe is a cocktail of probes.

22. The method of claim 1, wherein said at least one reporter probe is specific for a target nucleic acid sequence or antigen.

23. A kit for the detection of analytes in a sample comprising
a) at least one buffer;
b) liposomes, wherein said liposomes comprise an encapsulated amplificant and comprise at least one reporter probe on the surface of said liposomes;
c) at least one probe;
d) paramagnetic beads, wherein said paramagnetic beads comprise at least one capture probe;
e) a liposome extractant;
f) at least one luminescence reagent.

24. The kit of claim 23, wherein said encapsulated amplificant is selected from the group consisting of adenosine triphosphate (ATP), adenylate kinase (AK), luminol and a luciferase/luciferin cocktail.

25. The kit of claim 23, wherein said at least one reporter probe is a cocktail of probes.

26. The kit of claim 23, wherein said at least one reporter probe is specific for a target nucleic acid sequence or antigen.

27. The kit of claim 23, wherein said paramagnetic beads and said capture probe are labeled.

28. The kit of claim 27, wherein said paramagnetic beads are labeled with biotin and said capture probe is labeled with streptavidin.

29. The kit of claim 27, wherein said paramagnetic beads are labeled with streptavidin and said capture probe is labeled with biotin.

30. The kit of claim 23, wherein said luminescence reagent is selected from the group consisting of luciferase, luciferin and adenosine diphosphate; luciferase and luciferin; and ATP.

31. The kit of claim 23, wherein said liposomes are unilamellar or multilamellar.

32. The kit of claim 23, wherein the liposome extractant comprises a surface-active gluconate compound or derivative and an ethylene-amine compound or derivative.

33. The kit of claim 32, wherein the liposome extractant does not chemically and/or adversely affect subsequent detection reactions.

34. The kit of claim 23, further comprising a liposome extractant solution.

35. The kit of claim 23, further comprising a device comprising means for magnetic capture.

36. The kit of claim 23, further comprising a negative and positive control.

37. The kit of claim 23, further comprising written instructions for using said kit.
